(19) United States
(12) Patent Application Publication
(10) Pub. No.: US 2024/0110209 A1
(43) Pub. Date: Apr. 4, 2024

(54) METHOD TO PRODUCE MEDIUM CHAIN FATTY ACIDS

(71) Applicant: DRANCO, naamloze vennootschap, Gent (BE)

(72) Inventor: Jan Remi G. Smis, Merelbeke (BE)

(21) Appl. No.: 18/270,369

(22) PCT Filed: Jan. 4, 2022
(86) PCT No.: PCT/IB2022/050038

§ 371 Date: Jun. 29, 2023

(30) Foreign Application Priority Data
Jan. 4, 2021 (BE) 2021/5001

(51) Int. Cl. C12P 7/6409; C02F 11/04

(52) U.S. Cl. CPC C12P 7/6409; C02F 11/04

(57) ABSTRACT

A method to produce medium chain fatty acids from organic waste, contains the following steps: separating the organic waste into a solid fraction and a liquid fraction; fermenting the liquid fraction during a post-fermentation; separating the at least partly fermented liquid fraction by a biomass separation step into an effluent low in sludge and an effluent high in sludge; and extracting medium chain fatty acids from the effluent low in sludge.

### CROSS-REFERENCE TO RELATED APPLICATIONS

The present application is the National Phase entry of International Patent Application No. PCT/IB2022/050038 filed Jan. 4, 2022, which claims priority to Belgium Patent Application No. 2021/5001 filed Jan. 4, 2021, the entire contents of which are hereby incorporated by reference into this application.

### TECHNICAL FIELD

The present disclosure relates to a method to produce medium chain fatty acids.

More specifically it is a method to produce medium chain fatty acids from organic waste.

### BACKGROUND

Traditionally, medium chain fatty acids are extracted from coconut oil and palm oil.

A disadvantage of this is that relatively few medium chain fatty acids are obtained.

Another disadvantage is that coconut oil and palm oil form a non-sustainable source for the production of medium chain fatty acids.

The purpose of the present disclosure is to provide a solution to one of the aforementioned and other disadvantages.

### SUMMARY

To this end, the present disclosure relates to a method to produce medium chain fatty acids from organic waste, whereby the method contains the following steps:
fermenting organic waste in a main fermentation, whereby organic waste is at least partly converted into lactic acid;
separating organic waste into a solid fraction and a liquid fraction;
fermenting the liquid fraction during a post-fermentation, whereby at least a part of the lactic acid is converted into medium chain fatty acids;
separating the at least partly fermented liquid fraction by a biomass separation step into an effluent low in sludge and an effluent high in sludge;
extracting medium chain fatty acids from the effluent low in sludge.

An aspect of the present disclosure of this is that it is a sustainable method to produce medium chain fatty acids.

Another aspect of the present disclosure is that it is a sustainable and ecological method to process biological waste, whereby bioproducts are obtained with a higher added value (compared to classic waste treatment).

Another aspect of the present disclosure is that the method is an in situ production to produce medium chain fatty acids with, among other things, lactic acid as intermediate product.

The method allows organic waste to be processed into medium chain fatty acids without requiring external addition and/or in situ production of ethanol being necessary.

The lactic acid is less harmful and/or toxic than ethanol, and thus requires less strict safety measures and/or checks.

The separation of the organic waste into a solid fraction and a liquid fraction may be preceded by a mixing step, whereby the waste is mixed with a biomass.

When mixing the organic waste with the biomass, microorganisms and/or additives may also be added, for example enzymes and/or specific nutrients such as trace elements and/or minerals and/or vitamins and/or the like.

An aspect of the present disclosure is that the additives may stimulate the fermentation of the waste.

In some embodiments, the aforementioned mixing step is followed by a main fermentation, whereby the waste is fermented.

In case of a main fermentation and a post-fermentation, there may be a mixing step between the fermentation steps, whereby process water is added to the fermented mixture.

This process water can be obtained after separating the medium chain fatty acids from the effluent low in sludge.

During the post-fermentation and/or the main fermentation, process gas may be formed, the process gas may contain hydrogen gas in some embodiments.

The aforementioned hydrogen gas can be used in electrolytic production of protons.

An aspect of the present disclosure is that the method can be provided with the necessary substances for regulating the pH, without having to add external acids and/or bases.

The fermented mixture, with the medium chain fatty acids in solution, can be filtered by one or more membrane filters, whereby the biomass is retained and the medium chain fatty acids remain in the effluent low in sludge.

An aspect of the present disclosure is that the biomass can be reused, for example during the mixing of a biomass with the organic waste or during one or both fermentation steps.

The aforementioned method uses pre-existing installations such as tanks, reactors, presses, centrifuges, etc., such that the method is relatively simple to apply.

The different steps of the method may have an intermittent operation, whereby energy-absorbing steps can alternate, such that the power consumption can drop, which is advantageous both ecologically and economically.

In some embodiments, the method and the installations that can be used for this are provided with electricity by solar power and/or wind energy and/or biogas that can be obtained from the method.

This form of energy may also contribute to an electrolytic production of protons and/or hydroxide ions, which can be used to adjust the pH.

In some embodiments, only a limited amount of chemicals and/or water and/or biomass and/or the like are externally supplied.

The method can be provided with various internal recirculation possibilities to, for example, reuse chemicals and/or compounds and/or water and/or biomass and/or one or more combinations thereof and/or the like.

### BRIEF DESCRIPTION OF THE DRAWINGS

With the intention of better showing the characteristics of the present disclosure, a few applications of the method to produce medium chain fatty acids according to the present disclosure are described hereinafter by way of an example, without any limiting nature, with reference to the accompanying drawings, wherein:

FIG. 1 schematically shows the successive steps according to the present disclosure;

FIG. 2 shows an alternative method of FIG. 1.

### DETAILED DESCRIPTION

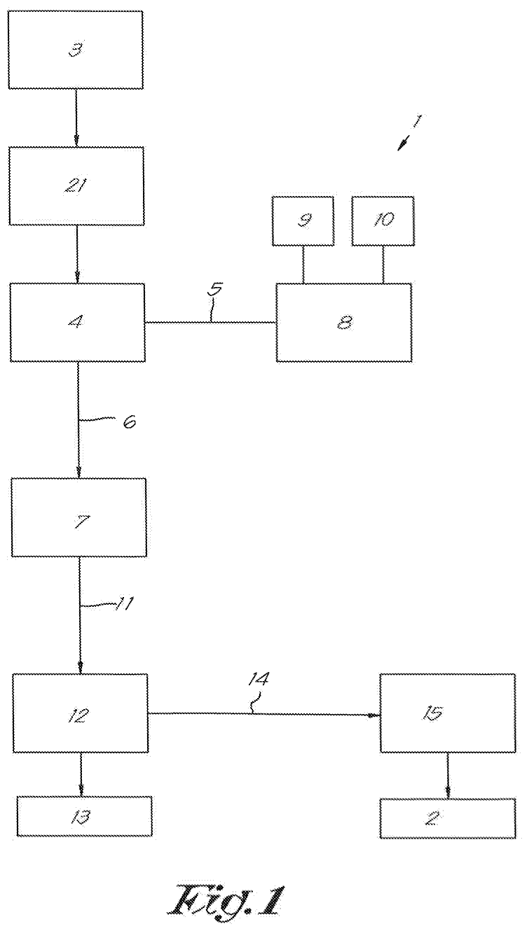

The method 1 shown in FIG. 1 is intended to produce medium chain fatty acids 2 from organic waste 3.

Medium chain fatty acids 2, are understood to mean fatty acids with at least six C-atoms, such as caproic acid, enanthic acid, octanoic acid, etc.

Different waste streams 3 are suitable to serve as substrate to produce such medium chain fatty acids 2, for example kitchen waste and/or food waste and/or other selectively collected simple decomposable organic waste 3 and/or other types of organic substrates with similar characteristics.

Such waste 3 may contain both liquid waste and solid waste or a combination thereof.

To this end, the organic waste 3 can be decomposed during the main fermentation 21 via hydrolysis and converted into lactic acid, which acts as intermediate product of the decomposition process and substrate for chain elongation.

During the main fermentation 21, lactic acid can partly be converted into intermediary products of the medium chain fatty acids production, for example short chain fatty acids such as acetic acid and butyric acid, and also into the end product medium chain fatty acids 2, including caproic acid.

The fermented organic waste 3 is separated 4 into a solid fraction 5 and a liquid fraction 6.

Such liquid fraction 6 may be obtained by separating 4 the waste 3 by sieves or presses or the like.

The liquid fraction 6 will then be fermented during a post-fermentation 7, whereby at least a part of the lactic acid is converted into medium chain fatty acids 2 as end product. In some embodiments, the aforementioned liquid fraction 6 still contains small organic particles, for example fibres or the like, said particles not being retained during sieving and/or pressing of the organic waste 3.

These organic particles can be converted during the post-fermentation 7 into lactic acid, after which at least a part of the lactic acid may be converted into medium chain fatty acids 2 during the post-fermentation 7.

Consequently, more medium chain fatty acids 2 can be obtained.

The solid fraction 5 can be converted into biogas 9 and/or compost 10 by anaerobic digestion 8.

In some embodiments, the known dry anaerobic composting (DRANCO) system is used for this.

Such post-fermentation 7 can take place in a continuous stirred tank reactor (CSTR), an upflow anaerobic sludge blanket reactor (UASB), a packed bed reactor (PBR) or the like.

After the post-fermentation 7, the fermented mixture 11 is separated by a biomass separation step 12, such as centrifugation and/or microfiltration, into an effluent high in sludge 13 which contains chiefly active microorganisms that can catalyse the fermentation 7 and into an effluent low in sludge 14 that is rich in medium chain fatty acids 2.

Separating the fermented mixture 11 is understood to mean separating the at least partly fermented liquid fraction by a biomass separation step 12.

The aforementioned effluent low in sludge 14 may also be sludge-free.

Subsequently, said effluent low in sludge 14 is used to extract 15 the medium chain fatty acids 2 from the effluent low in sludge 14.

This can be done by fluid-fluid extraction or electrochemical extraction 15, for example, whereby membrane filtration may or may not be used.

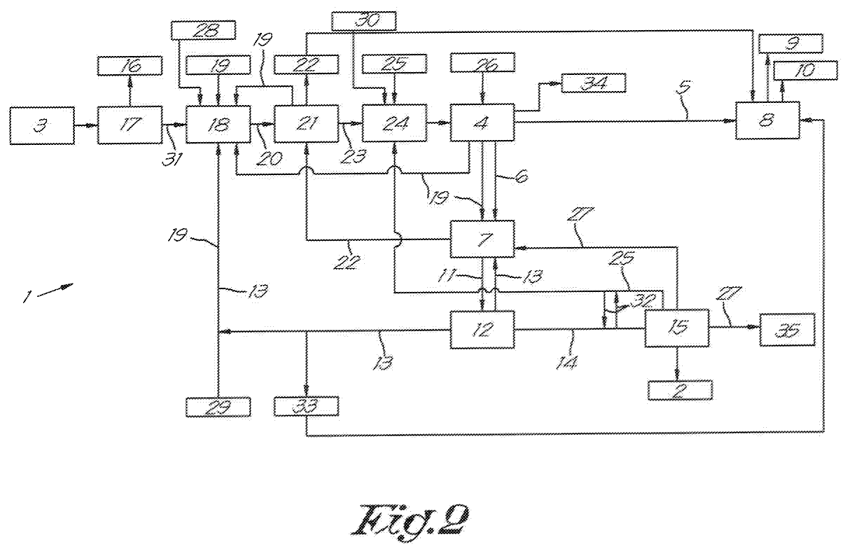

As shown in FIG. 2, in some embodiments the waste 3 is first pre-treated 17 to safeguard the waste 16 from any unwanted impurities, such as branches, stone, plastic, etc.

In this pre-treatment 17 of the waste 3, sieves can be used, among other things, which may or may not rotate and/or drums and/or the like.

In addition it is also possible to mechanically process the waste 3 during the pre-treatment 17, for example by crushing and/or chopping and/or shredding and/or the like, to stimulate the release of organic compounds.

In some embodiments, the organic waste 3 is mixed 18 with a biomass 19 under anaerobic conditions to form a mixture 20, the biomass 19 coming from a step further in the production process.

The method may be provided with different recirculation steps to be able to reuse the biomass 19.

Thus, biomass 19 may be obtained from the main fermentation 21 and/or after separating 4 in a solid fraction 5 and a liquid fraction 6 and/or after the biomass separation step 12, the obtained biomass 19 being able to be reused by adding it while mixing 18 the waste 3 or pre-treated waste 31.

The biomass 19 contains specific chain elongating microorganisms, such as Caproiciproducens sp and/or Ruminococcaceae sp and/or Clostridium kluyveri and/or Pseudoramibacter sp and/or lactic-acid-forming bacteria such as Lactobacillus sp and/or Olsenella sp and/or the like and can thus contribute to the production of medium chain fatty acids 2.

After mixing 18 the biomass 19 with the waste 3 or the pre-treated waste 31, the mixture 20 can be subjected to a main fermentation 21, whereby the mixture 20 is fermented under specific circumstances.

During said main fermentation 21, the organic waste 3 or the pre-treated waste 31 is at least partly fermented into lactic acid.

In some embodiments, the lactic acid that is produced during the main fermentation 21 is at least partly converted into medium chain fatty acids 2 during the main fermentation 21.

Additionally, during the main fermentation 21, process gas 22 can also be produced, the process gas 22 being able to be converted into methane.

In some embodiments, the process gas 22 is discharged to the anaerobic digestion 8 of the aforementioned solid fraction 5.

The aforementioned process gas 22 may contain hydrogen gas, said hydrogen gas being able to be added to the main fermentation 21 to stimulate the microbial chain elongation.

In some embodiments, the hydrogen gas is also used for the electrolytic production of protons, such that the method 1 can be provided with an internal acid supply.

During the main fermentation 21 and/or the post-fermentation 7, it is also possible ethanol is produced.

This possible in situ production of ethanol is not necessary to process the biological waste into lactic acid nor for the chain elongation of, for example, lactic acid into medium chain fatty acids 2.

Additionally, the method 1 is aimed at producing lactic acid which is subsequently converted into medium chain fatty acids 2 via microbial chain elongation.

In some embodiments, no large quantity of ethanol is produced and/or added during the main fermentation 21 and/or the post-fermentation 7.

After the aforementioned main fermentation 21, the fermented mixture 23 can be mixed 24, whereby process water 25 and/or fresh water 30 can be added to the mixture 23.

This process water 25 comes from a later step in the method 1 and can be obtained after the biomass separation step 12 and/or after the extraction step 15.

It is possible to indirectly discharge the process water 25 that can be obtained after the biomass separation step 12 via the extraction step 15 to the mixing step 24, whereby the fermented mixture 23 can then be mixed 24 with the discharged process water 25.

Between the biomass separation step 12 and the extraction step 15, a bypass 32 can be provided to let the aforementioned steps 12, 15 operate independently of each other.

In some embodiments, the process water 25 contains a relatively low concentration of medium chain fatty acids 2.

Fresh water 30 can be added if necessary or desired to bring or keep the concentrations of organic and inorganic compounds within the desired limits.

Fresh water 30 is understood to mean mains water, rainwater, groundwater and/or the like.

In some embodiments, after mixing 24 the process water 25 with the fermented mixture 23, the mixture 23 is separated 4 in at least the aforementioned liquid fraction 6 and solid fraction 5, whereby in this case too the liquid fraction 6 will be supplied to the post-fermentation 7.

It is also possible to separate 4 the organic waste 3 and/or the pre-treated waste 31 and/or the fermented mixture 23 into a liquid fraction 6, a solid fraction 5 and a fraction 19 rich in biomass.

The main fermentation 21 and/or the post-fermentation 7 are controlled such that medium chain fatty acids 2 with chiefly caproic acid is formed by the microbial chain elongation process.

To this end the organic waste 3 can be decomposed via hydrolysis during the main fermentation 21 and converted into lactic acid, which acts as an intermediate product of the decomposition process and substrate for chain elongation.

During the main fermentation 21, lactic acid can be partly converted into intermediary products of the medium chain fatty acids production, for example short chain fatty acids such as acetic acid and butyric acid, and into the end product medium chain fatty acids 2 including caproic acid.

During the production of lactic acid, the pH in the tank may drop, contrary to microbial chain elongation which has a pH increasing effect, such that the pH in the tank remains balanced.

Consequently, only a minimum regulation of the pH by, for example, protons or hydroxide ions and/or addition of acids and/or bases is needed.

During the post-fermentation 7, the liquid fraction 6 is fermented and the conditions in the reactor or tank are controlled such that the lactic acid and/or acetic acid and/or butyric acid that is still present in the reactor can be converted into medium chain fatty acids 2 such as caproic acid as most important end product.

In some embodiments, during the post-fermentation 7, a liquid waste 26, rich in lactic acid, can be added directly to the reactor.

However, as shown in FIG. 2, it is possible to first separate 4 the liquid waste 26, for example by sieves, after which the liquid fraction can be used for the post-fermentation 7.

The medium chain fatty acids production takes place during the aforementioned fermentation step or steps 7, 21 as long as the chain elongating organisms are not inhibited by the substrate or by the end product itself.

The aforementioned effluent high in sludge 13 can serve as biomass 19 that can be mixed 18 with the waste 3 or the pre-treated waste 31 before the main fermentation 21.

This effluent high in sludge 13 can be circulated multiple times between the post-fermentation 7 and the biomass separation step 12 to thus have control over the residence time of the sludge 33 in the tank of the post-fermentation 7.

In this case, the effluent low in sludge 14 contains a relatively large quantity of fatty acids in solution.

Thus, a wide range of carbon acids is present in the effluent low in sludge 14 such as: acetic acid, propionic acid, (iso)butyric acid, (iso)valeric acid, (iso)caproic acid, enanthic acid and/or caprylic acid.

As a result of a downstream extraction system 15 for medium chain fatty acids 2, chiefly caproic acid, enanthic acid and caprylic acid can be obtained as end product.

These aforementioned medium chain fatty acids 2 can thus be extracted 15 by fluid-fluid extraction or electrochemical extraction or the like, whereby the medium chain fatty acids 2 can also be extracted from the effluent low in sludge 14.

This extraction step 15 results in medium chain fatty acids 2, such as caproic acid and an aqueous solution 27 which is low in sludge and contains few to no medium chain fatty acids 2.

As shown in the example of FIG. 2 it is also possible to add one or more additives 28 during the mixing 18 of the organic waste 3 with the biomass 19, for example acids and/or bases and/or enzymes and/or one or more nutrients such as minerals, trace elements, vitamins.

Also, during the aforementioned mixing 18, an externally cultivated microbiome 29 can be added if the waste 3/biomass 19 ratio needs to be adapted to add a higher share of microbial biomass 19 to the system, in particular if it is desirable to adjust the share of chain elongating organisms.

In some embodiments, such cultivated microbiome 29 contains bacteria, such as Caproiciproducens sp and/or Ruminococcaceae sp and/or Clostridium kluyveri and/or Pseudoramibacter sp and/or lactic-acid-forming bacteria such as Lactobacillcus sp and/or Olsenella sp and/or the like.

Depending on the used reactor, the residence time of the biomass and of the liquid fraction 6 during the post-fermentation 7 may be set separately to be able to suppress any competitive reactions with microorganisms that show a slower growth, for example methanogens.

During the biomass separation step 12 the fermented mixture 11 can be filtered to be able to remove components that are bigger than 0.2 to 5 μm, whereby the medium chain fatty acids 2 remain present in the filtered effluent low in sludge 14.

It is not excluded that the effluent low in sludge 14 can be recirculated several times, for example to increase the yield of medium chain fatty acids 2 from the organic waste 3.

Thus, it is also possible to guide the short chain fatty acids in the effluent low in sludge 14 back to the post-fermentation 7, after the extraction 15 of the medium chain fatty acids 2, by an aqueous solution 27 such that said short chain fatty acids can still be converted into medium chain fatty acids 2.

The biomass 19 can also be reused after the biomass separation step 12, whereby the biomass 19 can be added to the tank of the post-fermentation 7 or mixed 18 with the organic waste 3 before the main fermentation 21.

In this way the incoming waste 3 and/or pre-treated waste 31 and/or mixture 20 and/or liquid fraction 6 can be inoculated with a significant quantity of active biomass 19, which can catalyse the fermentation 7, 21.

In this biomass separation step 12, a surplus of sludge 33 can be separated, the sludge 33 being able to be added during the anaerobic digestion 8.

This sludge 33 contains biomass 19, among others, such that a surplus of biomass 19 can be removed from the tanks and/or reactors.

During the post-fermentation 7, process gas 22 may be formed, said process gas 22 being able to be supplied to the main fermentation 7 and in this way ends up in the anaerobic digestion 8.

Alternatively, the formed process gas 22 is discharged from the tank of the post-fermentation 7 directly to the anaerobic digestion 8 of the solid fraction 5.

During the separation 4 of the waste 3 or the fermented mixture 23 in the solid fraction 5 and the liquid fraction 6, interfering substances 34 can also be separated and removed from the system.

In some embodiments, the temperature in the tank during the main fermentation 21 fluctuates between 25° C. and 60° C., between 30° C. and 50° C., or between 35° C. and 45° C., whereby microorganisms can grow under optimal conditions.

In some embodiments, the pH value in the tank during the main fermentation step 21 is at low operational pH-values between 4 and 6 and a dry matter content of 10-35%.

Typically, the main fermentation step 21 takes between 1 to 10 days.

In some embodiments, the post-fermentation 7 takes place between 6 hours to 10 days with a dry matter content of 1 to percent, 2 to 10 percent, or 4 to 8 percent.

In a practical embodiment, the temperature in the tank fluctuates during the post-fermentation 7 between 22° C. and 55° C., between 28° C. and 45° C., or between 30° C. and 37° C., whereby microorganisms are able to grow under optimal conditions.

Depending on the tank used during the post-fermentation 7, the residence time of the sludge 33 in the tank of the post-fermentation 7 can be set and if necessary the sludge 33 can be discharged to the anaerobic digestion 8.

This provides the aspect that any competitive reactions with the microorganisms such as methanogens can be suppressed.

In some embodiments, but not necessarily the pH values in the tank are higher during the post-fermentation 7 than during the main fermentation, for example with pH values between 5 and 7.

If necessary or desired, the pH value in the tank can be adjusted during both the post-fermentation 7 and the main fermentation 21.

In some embodiments, the pH value is lowered by adding protons that can be obtained from the process gas 22.

However, it is also possible to add an acid or base to the tank or reactor, if desired or necessary, to bring the pH value within the desired limits.

The present disclosure is not limited to the embodiments described as an example and shown in the figures, however, such methods can be realised according to different variants, without departing from the scope of the present disclosure.

1. A method to produce medium chain fatty acids from organic waste including kitchen waste and/or food waste and/or decomposable organic waste, the method contains the following steps:
fermenting the organic waste in a main fermentation, whereby the organic waste is at least partly converted into lactic acid;
separating the organic waste into a solid fraction and a liquid fraction;
fermenting the liquid fraction during a post-fermentation, whereby at least a part of the lactic acid is converted into medium chain fatty acids;
separating the at least partly fermented liquid fraction by a biomass separation step into an effluent low in sludge and an effluent high in sludge;
extracting medium chain fatty acids from the effluent low in sludge.

2. The method according to claim 1, wherein the main fermentation is preceded by mixing organic waste with a biomass to form a mixture.

3. The method according to claim 2, wherein when mixing the organic waste with the biomass, additives are added which stimulate the fermentation of the organic waste.

4. The method according to claim 3, wherein the additives contain acids and/or bases and/or enzymes and/or one or more nutrients such as minerals, trace elements, vitamins.

5. The method according to claim 1, wherein the main fermentation takes place at a relatively high dry matter content of between 10% and 35%.

6. The method according to claim 1, wherein the main fermentation takes place at temperatures between 25° C. and 60° C. in which microorganisms grow.

7. The method according to claim 1, wherein a pH value of the main fermentation is between 4 and 6 and is adjustable by adding an acid or a base or by protons and/or hydroxide ions.

8. The method according to claim 1, wherein the main fermentation takes a minimum of 1 day and a maximum of 10 days.

9. The method according to claim 1, wherein after the main fermentation a mixing step takes place to form an at least partly fermented mixture, whereby a process water is added to the at least partly fermented mixture.

10. The method according to claim 1, wherein the post-fermentation takes between 6 hours to 10 days.

11. The method according to claim 1, wherein the a pH value during the post-fermentation is between 5 and 7.

12. The method according to claim 1, wherein the post-fermentation takes place at a dry matter content between 1 and 15 percent.

13. The method according to claim 1, wherein the post-fermentation takes place at temperatures between 22° C. and 55° C. whereby microorganisms are able to grow.

14. The method according to claim 1, wherein extracting medium chain fatty acids from the effluent low in sludge comprises a fluid-fluid extraction or electrochemical extraction.

15. The method according to claim 2, wherein the mixture contains lactic acid and/or a substrate that are configured to be converted into lactic acid, the lactic acid and/or substrate that are configured to be converted during the main fermentation into intermediary products, such as short chain fatty acids, and into an end product medium chain fatty acids.

16. The method according to claim 1, wherein the organic waste or the fermented mixture contains lactic acid and/or short chain fatty acids which are configured to be converted during the post-fermentation into medium chain fatty acids as an end product.

17. The method according to claim 1, wherein lactic acid and/or the short chain fatty acids is converted into medium chain fatty acids, including caproic acid, during the post-fermentation and/or main fermentation by microbial chain elongation, whereby lactic acid and/or short chain fatty acids are configured to be produced as an intermediate product.

18. The method according to claim 1, wherein the solid fraction is converted into compost and/or biogas by anaerobic digestion.

19. The method according to claim 18, wherein during the post-fermentation and/or the main fermentation, process gas is obtained, the process gas being discharged directly and/or indirectly to the anaerobic digestion.

20. The method according to claim 2, wherein the organic waste is pre-treated before mixing with the biomass to form the mixture.
